(19) United States
(12) Patent Application Publication
(10) Pub. No.: US 2026/0152794 A1
(43) Pub. Date: Jun. 4, 2026

(54) EFFICACY OF CHRONIC PELVIC PAIN TREATMENT

(71) Applicant: THE UNIVERSITY COURT OF THE UNIVERSITY OF EDINBURGH, Edinburgh (GB)

(72) Inventors: Scott MACKENZIE, Edinburgh (GB); Andrew Wemyss HORNE, Edinburgh (GB); Lucy WHITAKER, Edinburgh (GB); Krina ZONDERVAN, Oxford (GB); Nilufer RAHMIOGLU, Oxford (GB)

(21) Appl. No.: 19/133,501

(22) PCT Filed: Nov. 28, 2023
(86) PCT No.: PCT/GB2023/053076

§ 371 Date: May 28, 2025

(30) Foreign Application Priority Data
Nov. 29, 2022 (GB) 2217921.2

(51) Int. Cl. C12Q 1/6883; C12Q 1/6858

(52) U.S. Cl. CPC C12Q 1/6883; C12Q 1/6858; C12Q 2600/106; C12Q 2600/156

(57) ABSTRACT

The present disclosure provides methods of determining whether or not to administer a subject gabapentin, said method comprising assessing the genetic profile of a subject considered for a gabapentin-based therapy and determining, on the basis of the genetic profile, whether or not to administer the subject gabapentin.

### FIELD

The present disclosure relates to novel genetic markers associated with response to gabapentin treatment and methods of use thereof.

### BACKGROUND

Chronic pelvic pain (CPP) in women is a debilitating condition with an estimated prevalence of 2-24% worldwide (1). Like other chronic pain conditions, CPP is associated with a marked reduction in quality of life and substantial economic cost (2). When causative pathology in CPP is identified (e.g., endometriosis), appropriate treatment can be initiated. However, at diagnostic laparoscopy, no underlying pathology is found in up to 55% of women (3). For women in whom no pathology is identified, learning of the idiopathic nature of their chronic pain and the lack of effective treatments can be difficult.

Although biological mechanisms driving laparoscopy-negative CPP are poorly understood, evidence suggests that like in other chronic pain conditions, central nervous system sensitisation may contribute (4). In addition, many women with CPP have a neuropathic element to their pain (5). This combined with the increased off-license prescribing of the neuromodulator gabapentin in CPP and the paucity of high-quality evidence supporting its use warranted further investigation through a large clinical trial (6). A trial investigating the effects of Gabapentin for CPP (GaPP2: Gabapentin for chronic pelvic pain in women trial) concluded that gabapentin was not superior to placebo in improving worst or average pain scores in women with CPP and was associated with more side-effects (7). However, in an embedded mechanistic study using functional MRI, gabapentin appeared to exert an effect on the anterior cingulate cortex in response to abdominal punctate stimuli (8). As functional MRI is currently a research modality only, there is an unmet need for another means of predicting the subset of women who may derive a therapeutic benefit from gabapentin.

Many epidemiological studies have shown that genetic variation contributes to chronic pain susceptibility in a variety of chronic pain phenotypes including CPP (9-11). Recent evidence has also shown that common genetic variants link chronic pain phenotypes and various neuroimaging traits (e.g., grey matter morphology) (12). Gabapentin has substantial inter-individual variability in both therapeutic response and vulnerability to adverse drug reactions. Variation in gabapentin efficacy is thought to be partially driven by pharmacokinetic factors such as dose-dependent oral bioavailability, drug transporter(s) activity and influence of age or renal function (13). For other mainly opioid analgesics, common genetic variants are associated with drug efficacy, toxicity, and risk of dependence (14, 15). Two studies have investigated gabapentin pharmacogenomics through a candidate SNP approach, identifying single nucleotide polymorphism(s) associated with anticonvulsant and analgesic treatment response (16).

At present, there are limited effective and evidence-based analgesic options for the treatment of CPP. It is amongst the objectives of the present disclosure to provide genetic markers that enable stratification of patients for a personalised medicine approach to treatment.

### SUMMARY

The present disclosure is based in part on the identification of genetic markers which are associated with a response to gabapentin or gabapentin efficacy.

In a first aspect, the present disclosure provides a method of determining whether or not to administer a subject gabapentin, said method comprising assessing the genetic profile of a subject considered for a gabapentin-based therapy and determining, on the basis of the genetic profile, whether or not to administer the subject gabapentin.

In another aspect, the disclosure provides a method of identifying subjects who are either more or less likely to respond to treatment with gabapentin, said method comprising assessing the genetic profile of a subject considered for a gabapentin based therapy and determining, on the basis of the genetic profile, whether or not that subject is more or less likely to respond to gabapentin.

The disclosure also provides a method of identifying subjects in whom gabapentin may be efficacious, said method comprising assessing the genetic profile of a subject considered for treatment with gabapentin and determining, on the basis of the genetic profile, whether or not gabapentin will be efficacious in that subject.

Also disclosed is a method of:
predicting a gabapentin response; or
stratifying individuals likely or not likely to benefit from gabapentin treatment;
said method comprising assessing the genetic profile of a subject considered for treatment with gabapentin and on the basis of the genetic profile either:
predicting whether or not gabapentin will be efficacious in that subject; or
stratifying the subject as likely or not likely to benefit from gabapentin treatment.

Without wishing to be bound by theory, the various methods and uses described herein are of of considerable benefit, as subjects in a gabapentin pain responder group where found to experience a significant reduction (e.g. an average 47% reduction) in average pain. Given the unfavourable side-effect profile of gabapentin and the risk of dependence, limiting its use in patients where it may have limited therapeutic effect is highly desirable.

The methods of the present disclosure may be applied to any suitable human or animal subject. For example, the subject may be any subject being considered for gabapentin treatment. The subject may be suffering from or predisposed/susceptible to a disease or condition which requires or could benefit from treatment with gabapentin. For example, the subject may be suffering from or predisposed/susceptible to chronic pain, such as neuropathic pain or chronic pelvic pain. The subject may be a human female patient suffering from (or predisposed/susceptible to) chronic pelvic pain.

The methods of the present disclosure may be performed in vitro or in vivo. The methods may be performed on a sample derived, provided or obtained from or by a suitable subject. The sample (provided by or obtained from a subject) may be subjected to a genetic profiling technique in order to establish the presence or absence of any of the genetic markers now known to be associated with a response to gabapentin or gabapentin efficacy.

Samples which may be subjected to the methods of the present disclosure may include, for example, samples which comprise or contain nucleic acid, especially genomic DNA. A sample, may comprise blood (including whole blood or a fraction thereof such as, for example, serum and/or plasma), tissue/glandular secretions, tissue scrapings, cells, biopsies, biological fluids (sweat, saliva, semen and the like), amniotic fluid, lymph, hair, nail, skin and bone samples. In one embodiment, the sample may comprise saliva and/or blood.

Accordingly, a method of this disclosure may comprise obtaining or providing a sample for genetic profiling, and making the necessary determination on the presence or absence of a particular genetic profile.

It should be noted that throughout this specification the terms “comprise” and/or “comprising” are used to denote that aspects and/or embodiments of the present disclosure “comprise” the noted features and as such, may also include other features. However, in the context of this disclosure, the terms “comprise” and “comprising” encompass embodiments in which the present disclosure “consists essentially of” the relevant features or “consists of” the relevant features.

It should also be understood that this specification makes reference to genetic markers which have been shown to be associated with a “response to gabapentin” or with “gabapentin efficacy”. The term ‘response’ embraces not only an improved response to gabapentin, but also a reduced or null (i.e. no) response to gabapentin. Any level of gabapentin response would be assessed relative to a control, standard or known level of gabapentin response. For example, a control gabapentin response level may be that occurring in a subject having an established or known genetic profile (especially at the various loci described herein). Similarly, the term “gabapentin efficacy”, includes an increase or decrease (e.g. reduction) in gabapentin efficacy as compared to some control, known or standard level of gabapentin efficacy. Without being bound to any particular method, a control, standard or known level of gabapentin efficacy level may be that occurring in a subject having a known or established genetic profile (especially at the various loci described herein).

Additionally, the step of performing a “genetic profiling” technique on a sample involves probing the nucleic acid in that sample for the presence or absence of one of more genetic markers—including the genetic markers detailed herein.

A genetic marker may be identified or detected by any suitable profiling method or technique. One of skill will be familiar with polymerase chain reaction (PCR) or restriction enzyme based techniques and other sequencing technologies which may be used to determine the presence of one or more genetic markers in a sample. The skilled person in the art would also recognise that alternative techniques, such as next generation sequencing methods, may be employed to identify or detect one or more genetic markers.

Genetic markers to be exploited in the methods of the present disclosure may comprise, for example “variant” nucleic acid and/or “variant” gene sequences—the variant sequences being associated with a particular (improved or null) response to gabapentin treatment or gabapentin efficacy.

The variant sequences may be present at one or more different chromosomal loci.

Any given nucleic acid or gene sequence may be regarded as a “variant” sequence if, relative to, for example, a corresponding reference sequence, the ‘variant’ sequence possesses one or more sequence addition(s), deletion(s), substitution(s) and/or inversion(s). A corresponding reference sequence may comprise a “wild-type” sequence or a sequence known to be associated with a poor, normal or standard response to gabapentin treatment. A variant sequence of this disclosure may be present within a gene sequence (thus the gene may be regarded as a variant gene) and/or an intergenic region. Sequence variations to be exploited in the methods of this disclosure may take the form of one or more polymorphism(s), where, for example, individual nucleotides are substituted for nucleotides not present in the wild-type or reference sequence. In other embodiments, a “variant” nucleic acid sequence may result from the occurrence of one or more mutations within the sequence. Again, as one of skill will appreciate, a mutated nucleic acid sequence may comprise one or more nucleotide inversion(s), addition(s), deletion(s) and/or substitution(s).

As such, the present disclosure provides methods in which subjects who may respond more or less, to treatment with gabapentin, are identified on the basis of the presence or absence of one or more sequence polymorphisms, for example single nucleotide polymorphisms (SNPs). Those polymorphisms or SNPs may occur at one or more specific genetic loci. As such, if any of the methods described herein reveal that a subject harbours one or more of the alleles disclosed herein at one or more of the specific sequence polymorphism(s) (including SNP) loci now known to be associated with gabapentin response/efficacy, then that subject may be identified as one who either should or should not be administered gabapentin. The skilled reader will understand that the actual decision (whether to administer gabapentin or not) will depend on which SNP alleles have been identified as present or absent.

It should be understood that the phrase “variant sequence(s) associated with a response to gabapentin treatment/efficacy” encompasses any link or correlation between the presence and/or absence of one or more specific allele(s) (for example in the form of a specific allele at a specific polymorphic/SNP locus) and a response to gabapentin treatment and/or gabapentin efficacy. For example, any of the disclosed variant sequences may be associated with a good response to gabapentin treatment or with gabapentin efficacy.

There follows a summary of some of the genetic markers found to be associated with or indicative of, a response to gabapentin treatment or gabapentin efficacy. The methods provided by the present disclosure may be based on the assumption that the presence of one or more of the sequence variations described herein, indicates that a subject is likely to respond to gabapentin treatment. In other words, the sequence variations described herein may be markers of subjects (i) who are gabapentin responders and (ii) in whom gabapentin is efficacious.

Again, an ‘improved gabapentin response’ or ‘improved gabapentin efficacy’ is an assessment made relative to a control gabapentin response level or control gabapentin efficacy in (for example) a subject (i) not harbouring all, or at least some, of the genetic markers disclosed herein, (ii) known not to respond to gabapentin and/or (iii) in whom the gabapentin response or efficacy is not significantly different from placebo.

The following sections use the terms “variations associated” with a particular gene(s). The term “associated” is intended to embrace not only variations within sequences that are operatively linked with a specific gene or genes (for example within promoter elements and the like) but also variations regarded as within the “recombination area” or “recombination zone” of a particular gene or genes. Sequences associated with a particular gene or genes may be intergenic sequences—those being sequences which lie between gene sequences. For brevity, the variant sequences embraced by the present disclosure (intergenic or gene sequence variations, polymorphisms, SNPs and the like) shall be broadly referred to as “variant [gene] sequences” and “variant [gene] associated sequences” where the term “[gene]” is substituted for any of the specific genes described herein.

In particular, the present disclosure relates to the identification of variant sequences and/or polymorphisms (e.g. polymorphic loci) on human chromosome 10.

More specifically, the disclosure relates to variant sequences and/or polymorphisms (e.g. polymorphic loci) at or within the chromosomal loci 10q23 (10q23.1). These sequence variations are correlated with gabapentin response and gabapentin efficacy and therefore are important considerations in the management of pain, including chronic pelvic pain.

In one embodiment, a method of the present disclosure may comprise determining whether or not any of the 10q23 (10q23.1) variant sequences disclosed herein (and which are correlated with an improved response to gabapentin) are present in a sample obtained from, or provided by a subject, wherein if the subject is found to harbour one or more of the disclosed 10q23 (10q23.1) variant sequences, they may or may not be prescribed or administered gabapentin.

The present disclosure provides sequence variations within, associated with or proximal to the Neuregulin 3 (NRG3) gene on chromosome 10q23 (10q23.1). These NRG3 sequence variations have been shown to be associated with a response to gabapentin treatment or gabapentin efficacy. Specifically, the inventors have discovered that sequence variations within this gene and/or the associated recombination area (that is the recombination area 5′ and/or 3′ (upstream or downstream) to the gene) are associated with an improved response to gabapentin treatment or improved gabapentin efficacy.

Further, in some embodiments, subjects possessing or harbouring genetic variations on chromosome 10q23.1 may exhibit different responses to gabapentin treatment. Specifically, the present disclosure establishes a correlation/association between variants on chromosome 10q23.1 and/or variants in linkage disequilibrium with NRG3 gene, and response to gabapentin treatment.

In the context of this disclosure, a specific genetic locus (a ‘marker locus’) is “associated with” another locus, when the relevant loci are part of the same linkage group, due to physical chromosomal association, and are in linkage disequilibrium. This occurs when the marker locus and a linked locus are found together in progeny more frequently than if the loci segregate randomly. Similarly, a marker locus can also be associated with a trait, e.g., a marker locus can be “associated with” a given trait (in this case an improved response to gabapentin treatment or gabapentin efficacy) when the marker locus is in linkage disequilibrium with the trait (this can be detected, e.g. when the marker is found more commonly in ‘case’ versus ‘control’ populations). The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random frequency (in the case of co-segregating traits, the loci that underlie the traits are in sufficient proximity to each other). Linked loci co-segregate more than 50% of the time, e.g., from about 51% to about 100% of the time. Advantageously, the two loci are located in close proximity such that recombination between homologous chromosome pairs does not occur between the two loci during meiosis with high frequency, e.g., such that closely linked loci co-segregate at least about 80% of the time, more preferably at least about 85% of the time, still more preferably at least 90% of the time, e.g., 91%, 92%, 93%, 94%, 95%, 96%, 97%, 98%, 99% or more of the time.

Thus the present disclosure provides methods which exploit variant NRG3 sequences and/or variant NRG3 associated sequences. For the avoidance of doubt, the term “variant NRG3 sequences” encompasses intergenic or gene sequence variations and any polymorphisms, single nucleotide polymorphism(s) and the like occurring within the NRG3 gene. In a preferred embodiment, the term “variant NRG3 sequences” and/or “variant NRG3 associated sequences” comprise single nucleotide polymorphism(s) occurring within the NRG3 gene or within loci proximal to or associated with the NRG3 loci.

Without wishing to be bound by theory, NRG3 and variant forms thereof have, for the first time been associated with the response to gabapentin treatment and/or with gabapentin efficacy. For convenience, hereinafter, this gene (and any other associated with response to gabapentin) shall be referred to as “gabapentin response associated gene(s)”. A gabapentin response associated gene may be any gene the expression, function and/or activity of which is linked to or associated with a level of response to gabapentin treatment or gabapentin efficacy.

Again, without wishing to be bound by theory, it is suggested that sequence variations found to be associated with gabapentin response and/or efficacy may directly or indirectly affect or modulate the function, activity and/or expression of one or more gabapentin associated gene(s). It is suggested that modulation of expression, function and/or activity of a gabapentin associated gene (for example NRG3), might lead to (or contribute to, or be the cause of) the observed modulation of the gabapentin response/efficacy.

As stated, the disclosed sequence variations may take the form of one or more polymorphism(s) (i.e. SNPs) and the details of certain, specific SNPs (found to be linked to, associated with or indicative of a modulated (for example improved) response to gabapentin treatment) are detailed herein (Table 1). One of skill will appreciate that the present disclosure may further embrace other (sequence) variations, polymorphisms and/or SNPs which are themselves associated with the SNPs identified in Table 1. For example, the present disclosure may embrace SNPs which are identified as being in linkage disequilibrium with any one of the SNPs detailed in Table 1. GRCh37 (genome Reference Consortium Human Build 37) is the reference genome used herein to refer to the chromosome: position of the SNPs.

TABLE 1
SNPs of genome-wide significance for gabapentin pain response.

| | | | | | EAF in | EAF in | | | |
|---|---|---|---|---|---|---|---|---|---|
| | | | | | gabapentin | gabapentin | | | |
| | Position | | | | pain | pain non- | | | OR (95% |
| Chromosome | (GRCh37) | EA | OA | EAF | responders | responders | P value | Beta | CI) |
| 10 | 84685978 | T | G | 0.583 | 0.812 | 0.424 | 2.10E−08 | 2.94 | 18.82 |
| | (rs4442490) | | | | | | | | (4.86- |
| | | | | | | | | | 72.83) |
| 10 | 84690540 | T | C | 0.660 | 0.899 | 0.495 | 4.10E−08 | 2.63 | 13.84 |
| | (rs4933859) | | | | | | | | (4.34- |
| | | | | | | | | | 44.12) |
| 10 | 84681635 | T | G | 0.659 | 0.899 | 0.494 | 4.20E−08 | 2.62 | 13.75 |
| | (rs10885506) | | | | | | | | (4.32- |
| | | | | | | | | | 43.71) |
| 10 | 84685529 | A | G | 0.660 | 0.899 | 0.494 | 4.20E−08 | 2.62 | 13.76 |
| | (rs1923561) | | | | | | | | (4.33- |
| | | | | | | | | | 43.77) |
| 10 | 84688752 | A | C | 0.660 | 0.899 | 0.494 | 4.20E−08 | 2.62 | 13.76 |
| | (rs2026495) | | | | | | | | (4.33- |
| | | | | | | | | | 43.75) |
| 10 | 84682591 | C | G | 0.660 | 0.899 | 0.494 | 4.20E−08 | 2.62 | 13.75 |
| | (rs1923565) | | | | | | | | (4.32- |
| | | | | | | | | | 43.73) |

EA: effect allele; OA: other allele; EAF: effect allele frequency; OR: odds ratio; CI: confidence interval.

Thus the present disclosure provides a method of assessing a subject's likely response to gabapentin, the method comprising determining the allele present at one or more of the specific SNPs located at position(s) selected from the group consisting of:
(i) chr10:84685978 (rs4442490);
(ii) chr10:84690540 (rs4933859);
(iii) chr10:84681635 (rs10885506);
(iv) chr10:84685529 (rs1923561);
(v) chr10:84688752 (rs2026495);
(vi) chr10:84682591 (rs1923565); and/or
(vii) SNPs or genetic variations in linkage disequilibrium with any of (i)-(vi);
wherein depending on the allele present at one or more of the above-listed SNP positions, the subject may or may not respond to treatment with gabapentin. For example, where the allele present at any of the given SNP loci is determined as follows:
(i) chr10:84685978=T
(ii) chr10:84690540=T
(iii) chr10:84681635=T
(iv) chr10:84685529=A
(v) chr10:84688752=A
(vi) chr10:84682591=C;
the subject may respond to treatment with gabapentin and may be offered a gabapentin-based therapeutic option.

In contrast, where the allele present at any of the given SNP loci is determined as follows:
(i) chr10:84685978=G;
(ii) chr10:84690540=C;
(iii) chr10:84681635=G;
(iv) chr10:84685529=G;
(v) chr10:84688752=C;
(vi) chr10:84682591=G;
the subject may not respond to treatment with gabapentin and may be offered an alternative therapeutic option.

In view of the above, the inventors have discovered that certain SNP profiles are associated with an effective or improved response to gabapentin whereas other SNP profiles are associated with a poor or reduced response to gabapentin. The following SNP profiles have been identified as “responder” SNP profiles—in other words, subjects harbouring one or more of the following SNP profiles may be classed as subjects more likely to respond to gabapentin treatment (i.e. they are gabapentin responders).

| SNP reference | Gabapentin responder SNP alleles |
|---|---|
| chr10: 84685978 | T |
| chr10: 84690540 | T |
| chr10: 84681635 | T |
| chr10: 84685529 | A |
| chr10: 84688752 | A |
| chr10: 84682591 | C |

By way of example, for SNP chr10:84685978, the “responder” allele is “T”. A sample from a patient found to harbour this specific SNP allele would be identified or classed as a (potential) gabapentin responder and should be considered for gabapentin therapy.

In addition, the following SNP profiles have been identified as “non-responder” SNP profiles—in other words, subjects harbouring one or more of the following SNP profiles may be classed as subjects less likely to respond to gabapentin treatment (i.e. they are gabapentin non-responders).

| SNP reference | Gabapentin non-responder SNP alleles |
|---|---|
| chr10: 84685978 | G |
| chr10: 84690540 | C |
| chr10: 84681635 | G |
| chr10: 84685529 | G |
| chr10: 84688752 | C |
| chr10: 84682591 | G |

As stated, the results of any of the methods described herein may be compared to control or reference data. Useful control or reference data may comprise the results of SNP analysis performed on subjects exhibiting a known or calibrated response to gabapentin treatment. The reference or control data may comprise SNP and/or sequence profiles obtained from subjects with a known response to gabapentin.

In addition to the gene (SNP) based profiling methods described herein, the present disclosure may further extend to methods which probe, investigate or determine the function, expression and/or activity of any of the gabapentin response associated gene sequences disclosed herein. One of skill will appreciate that any modulation of a gabapentin response associated gene sequence may affect the magnitude or level of any response to gabapentin treatment in a subject. As stated above, the gabapentin response associated variants may include variants in the NRG3 gene (NRG3 variants), variants in linkage disequilibrium with the NRG3 variants and/or variants within or proximal to the NRG3 gene. “Proximal to” may refer to variants within 200 kb, 150 kb, 100 kb, 80 kb, 60 kb, 50 kb, 40 kb, 30 kb, 10 kb, 5 kb or fewer of relevant locus.

Without wishing to be bound by theory, it is suggested that variant NRG3 genes (which variant genes may harbour one or more of the disclosed SNP alleles) may further exhibit altered or modulated levels of expression. For example the presence of one or more of the specific SNP alleles described herein may result in a modulated (for example reduced or increased) level of NRG3 expression which in turn leads to a modulated (reduced or increased) level of neuregulin 3 expression. A level of neuregulin 3 expression may therefore also be associated with a response to gabapentin or gabapentin efficacy.

The term “modulated” function, expression and/or activity may embrace any increase or decrease in gene function, expression and/or activity relative to the function, expression and/or activity of the same gene in a control system, sample or subject. As stated, a control system, sample or subject may be any system, sample or subject associated with a particular (and known or calibrated) level of response to gabapentin treatment.

Samples which may be subject to methods which determine a level of expression, function and/or activity of a gabapentin response associated gene are listed above and include, for example, any sample which comprises nucleic acid including blood (whole blood), serum, plasma, secretions, biopsies, biological fluids (e.g. saliva and lymph) and the like.

The methods which may be used to detect modulated expression, function and/or activity of gabapentin response associated gene, or indeed a variant nucleic acid sequence (for example a SNP sequence) as disclosed herein, may include, polymerase chain reaction (PCR) based techniques utilising, for example using genomic DNA as template or reverse transcriptase (RT)-PCR (see below) techniques in combination with real-time PCR (otherwise known as quantitative PCR).

Other useful techniques may include restriction fragment length polymorphism analysis (RFLP) and/or hybridisation techniques using probes and/or primers designed to hybridise under conditions of high, medium and/or low stringency, to sequences within these loci. Suitable probes and/or primers (i.e. oligonucleotide sequences) are described herein (see below). Further information on such techniques may be found in Molecular Cloning: A Laboratory Manual (Third Edition) By Sambrook, MacCallum & Russell, Pub. CSHL; ISBN 978-087969577-4—incorporated herein by reference.

For example, PCR techniques useful in the detection of specific sequences (including specific SNP sequences) may require the use of short oligonucleotide primers designed to hybridise to sequences proximal to (for example 3′ and 5′ (upstream or downstream)) of a nucleic acid sequence of interest—for example a sequence potentially harbouring a SNP as disclosed herein. Once oligonucleotides have been hybridised to a nucleic acid sequence, the nucleic acid sequence between the primers is enzymatically amplified via the PCR. The amplified nucleic acid may then be sequenced to determine whether or not it comprises a variant sequence and/or the SNP of interest.

Additionally, or alternatively, the amplified nucleic acid may be contacted with one or more restriction enzymes—this technique is particularly useful if a variant nucleic acid sequence of SNP is known either to remove a particular restriction site or create a restriction site. The presence or absence of a variant nucleic acid sequence may be detected via analysis of the resulting restriction fragment length polymorphism (RFLP) profile.

When analysing RFLP profiles, the results may be compared to standard or control profiles obtained by contacting nucleic acid sequences obtained from subjects with a known or calibrated level of gabapentin response, with the same restriction enzymes.

In addition to the above, altered electophoretic mobility may be used to detect variations or alterations in nucleic acid sequences. For example, small sequence deletions and insertions may be visualised by high resolution gel electrophoresis—nucleic acid sequences with different sequences migrating through agarose gels (denaturing or non-denaturing and/or gradient gels) at different speeds/rates.

Relative levels of mRNA expression may be used as a means of determining the level of expression, activity and/or function of a particular gene (such as, for example, the gabapentin responder associated genes described herein). By way of example, modulation (i.e. an increase or decrease) in the amount of mRNA encoding the genes now found to be associated with a gabapentin response, may indicate modulated gene expression, function and/or activity and may further indicate that a subject is, or is not, likely to respond to gabapentin treatment.

More specifically, real time-PCR may be used to determine the level of expression of any of the gabapentin response associated markers described herein. Typically, and in order to quantify the level of expression of a particular nucleic acid sequence, RT-PCR may be used to reverse transcribe the relevant mRNA to complementary DNA (cDNA). Preferably, the reverse transcriptase protocol may use primers designed to specifically amplify a sequence of interest. Thereafter, PCR may be used to amplify the cDNA generated by reverse transcription. Typically, the cDNA is amplified using primers designed to specifically hybridise with a certain sequence and the nucleotides used for PCR may be labelled with fluorescent or radiolabelled compounds.

One of skill in the art will be familiar with the technique of using labelled nucleotides to allow quantification of the amount of DNA produced during a PCR. Briefly, and by way of example, the amount of labelled amplified nucleic acid may be determined by monitoring the amount of incorporated labelled nucleotide during the cycling of the PCR.

Further information regarding the PCR based techniques described herein may be found in, for example, PCR Primer: A Laboratory Manual, Second Edition Edited by Carl W. Dieffenbach & Gabriela S. Dveksler: Cold Spring Harbour Laboratory Press and Molecular Cloning: A Laboratory Manual by Joseph Sambrook & David Russell: Cold Spring Harbour Laboratory Press.

Additionally, or alternatively, a level of gene expression may be identified by way of microarray analysis. Such a method would involve the use of a DNA micro-array which comprises nucleic acid derived from gabapentin response associated genes. To identify a level gene expression, one of skill in the art may extract the nucleic acid, preferably the mRNA, from a sample and subject it to an amplification protocol such as, RT-PCR to generate cDNA. Preferably, primers specific for a target mRNA sequence—in this case sequences encoding any gabapentin response associated gene may be used.

Any amplified cDNA may be subjected to a further amplification step, optionally in the presence of labelled nucleotides (as described above). Thereafter, the optionally labelled amplified cDNA may be contacted with the microarray under conditions which permit binding with the DNA of the microarray. In this way, it may be possible to identify a level of gabapentin response associated gene expression.

In addition, other techniques such as deep sequencing and/or pyrosequencing may be used to detect variant sequences which are associated with the gabapentin response in a subject. Further information on these techniques may be found in “Applications of next-generation sequencing technologies in functional genomics”, Olena Morozovaa and Marco A. Marra, Genomics Volume 92, Issue 5, November 2008, Pages 255-264 and “Pyrosequencing sheds light on DNA sequencing”, Ronaghi, Genome Research, Vol. 11, 2001, pages 3-11.

In other embodiments, samples provided by subjects to be tested may be analysed or probed for the levels of the protein products of the gabapentin response associated variants described herein. For convenience, these protein products shall be referred to as “gabapentin response associated proteins”—this would include the protein product of the NRG3 gene—neuregulin 3. One of skill will appreciate that the level of protein production may be correlated with the level of expression of the corresponding gene and thus a level of gabapentin response associated protein expression may be associated with a level of gabapentin response associated gene expression. As such, the level of expression of any gabapentin response associated protein may be used to gauge, determine or assess the likelihood that a particular subject will respond to gabapentin treatment.

Immunological detection techniques such as, for example, enzyme linked immunosorbent assays (ELISAs) or Western blot and/or immunoblot techniques may be used to detect levels of gabapentin response associated protein in any of the samples (or directly in a subject) described herein. Such techniques may require the use of binding agents or antibodies specific to, or selective for, the various gabapentin response associated gene products (or (immunogenic/functional/epitope containing) fragments thereof) described herein. Further information on such techniques may be found in Using Antibodies: A Laboratory Manual By Harlow & Lane, Pub. CSHL, ISBN 978-087969544-6 and Antibodies: A Laboratory Manual by Harlow & Lane, CSHL, ISBN 978-087969314-5-both of which are incorporated herein by reference.

Other techniques which exploit the use of agents (for example antibodies) capable of binding gabapentin associated proteins, include, for example, techniques such as western blot or dot blot. A western blot may involve subjecting a sample to electrophoresis so as to separate or resolve the components, for example the proteinaceous components, of the sample. The resolved components/proteins may then be transferred to a substrate, such as nitrocellulose.

To identify any gabapentin response associated proteins present in a sample, the substrate (for example nitrocellulose substrate) to which the resolved components and/or proteins have been transferred, may be contacted with a binding agent capable of binding gabapentin response associated proteins under conditions which permit binding between any gabapentin response associated proteins present in the sample (or transferred to the substrate) and the agents capable of binding the gabapentin response associated proteins.

Advantageously, the agents capable of binding the gabapentin response associated proteins may be conjugated to a detectable moiety.

Additionally, the substrate may be contacted with a further binding agent having affinity for the binding agent(s) capable of binding gabapentin response associated proteins. Advantageously, the further binding agent may be conjugated to a detectable moiety.

Other immunological techniques which may be used to identify a level of gabapentin response associated protein in a sample (particularly tissue or biopsy samples) include, for example, immunohistochemistry wherein gabapentin response associated protein binding agents, are contacted with a sample such as those described above, under conditions which permit binding between any gabapentin response associated protein present in the sample and the binding agent. Typically, prior to contacting the sample with the binding agent, the sample is treated with, for example a detergent such as Triton X-100. Such a technique may be referred to as “direct” immunohistochemical staining. The level of gabapentin response associated protein binding agent binding may be used as a measure of the level of gabapentin associate protein present in a sample.

To help quantify or identify the levels of gabapentin response associated protein present in a sample, one may compare the results of any of the immunological of molecular (e.g. PCR, RT-PCR) techniques described herein, with results obtained from the same procedures using control or reference samples obtained from subjects having known or calibrated gabapentin responses.

In a further aspect the present disclosure, provides polynucleotide/oligonucleotide probes or primers useful in the detection of modulated expression of any of the gabapentin response associated markers described herein and/or any of the SNPs of the present disclosure. Polynucleotide sequences of the present disclosure may comprise nucleotide sequences which are complementary to nucleotide sequences (preferably contiguous sequences) adjacent to and/or comprising, the variant sequences described herein. The polynucleotide sequences may be primer or probe sequences which may otherwise be referred to as oligonucleotides. In one embodiment, the probe or primer oligonucleotides provided by the present disclosure may comprise, for example 5-50, 6-40, 7-30, 8-20 nucleotides. In one embodiment, and particularly where the oligonucleotide has application as a probe, the oligonucleotide may comprise a nucleotide complementary to regions adjacent a particular SNP. One of skill will appreciate that primer sequences comprising nucleotides complementary to sequences upstream and/or downstream of any of the SNPs described herein, may be used in PCR based techniques to amplify sections of nucleic acid comprising one or more SNP or SNP loci. Oligonucleotide sequences useful as probes or primers may be used to detect the presence or absence of certain SNP sequences in nucleic acid samples provided by subjects.

It should be noted that gene(s) homologous to the human gabapentin response associated gene NRG3, may be found in a number of different species, including, for example, other mammalian species. Homologous gene(s) may exhibit as little as approximately 20 or 30% sequence homology or identity however, in other cases, homologous genes may exhibit at least 40, 50, 60, 65, 70, 75, 80, 85, 90, 91, 92, 93, 94, 95, 96, 97, 98, 99% homology to the various nucleotide sequences given above. As such, homologous genes from other species and equivalent methods based thereon are to be included within the scope of the present disclosure.

A further aspect of the present disclosure relates to kits for identifying subjects more or less likely to respond to treatment with gabapentin. A kit according to this aspect of the present disclosure may include one or more pairs of oligonucleotide primers useful for amplifying a nucleotide sequence of interest. For example, the nucleotide sequence of interest may comprise one or more nucleic acid sites known to harbour the variant, or SNP sequences disclosed herein. The kit may comprise a polymerizing agent, for example, a thermo-stable nucleic acid polymerase such as one disclosed in U.S. Pat. No. 4,889,818 or 6,077,664. Furthermore, the kit may comprise an elongation oligonucleotide that hybridizes to sequence adjacent or proximal to a site potentially harbouring a variant sequence or SNP of the present disclosure. Where the kit includes an elongation oligonucleotide, it may also include chain elongating nucleotides, such as dATP, dTTP, dGTP, dCTP, and dITP and/or analogues thereof. In addition, the kit provided by this aspect of the present disclosure may optionally include terminating nucleotides such as, for example, ddATP, ddTTP, ddGTP, ddCTP. The kit may include one or more oligonucleotide primer pairs, a polymerizing agent, chain elongating nucleotides, at least one elongation oligonucleotide, and one or more chain terminating nucleotides. Kits may also optionally include reaction and/or storage buffers, vials or other storage/reaction vessels, microtitre plates and instructions for use.

### DETAILED DESCRIPTION

The present disclosure is described with reference to the following figures, which show:

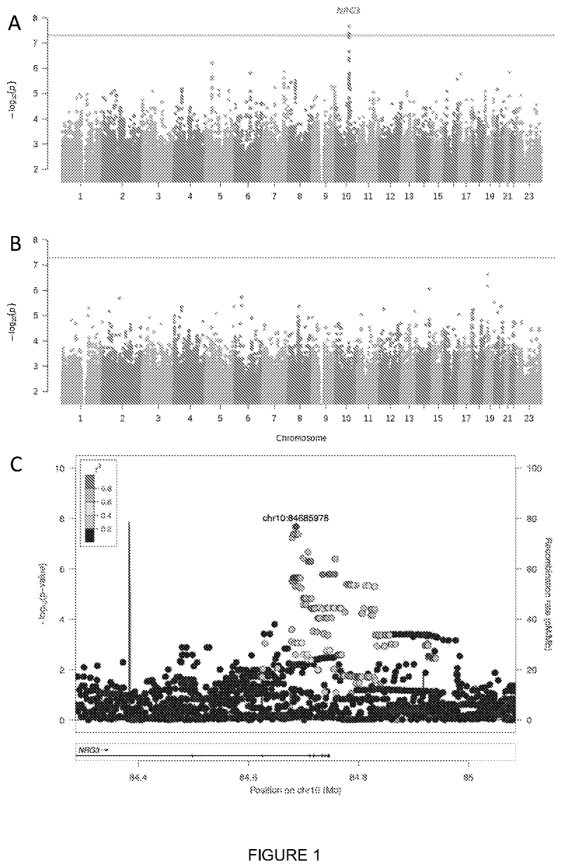

FIG. 1. Manhattan plots and regional association plot of GWAS results. A) Manhattan plot of gabapentin pain response; B) Manhattan plot of side-effect burden. C) Regional association plot gabapentin pain response lead SNP (chr10:84685978), generated using LocusZoom (http://locuszoom.org/). The genome-wide significance threshold (p<5×10-8) is indicated by the red horizontal line.

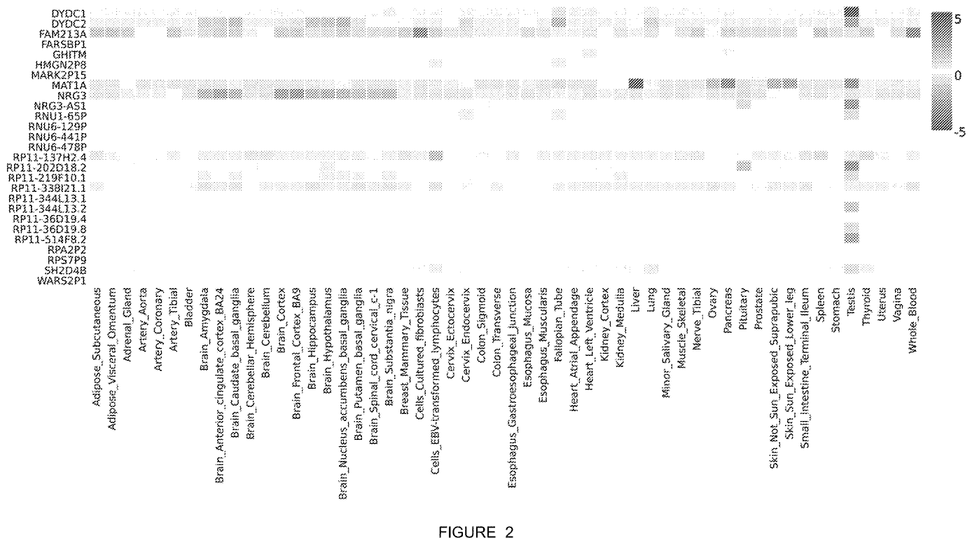

FIG. 2. Bulk gene expression in GTEx v8 of mapped genes from FUMA GENE2FUNC (average of normalised expression per label (log 2 transformed)).

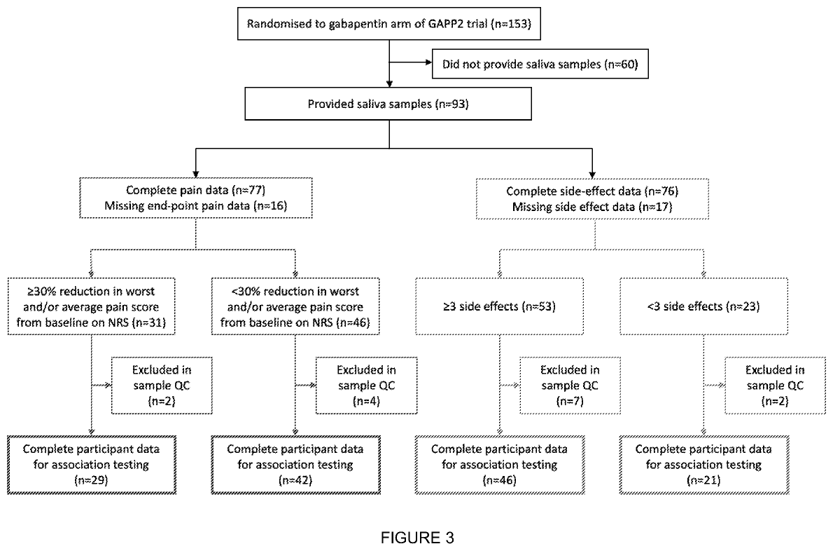

FIG. 3: A study flowchart is of participants.

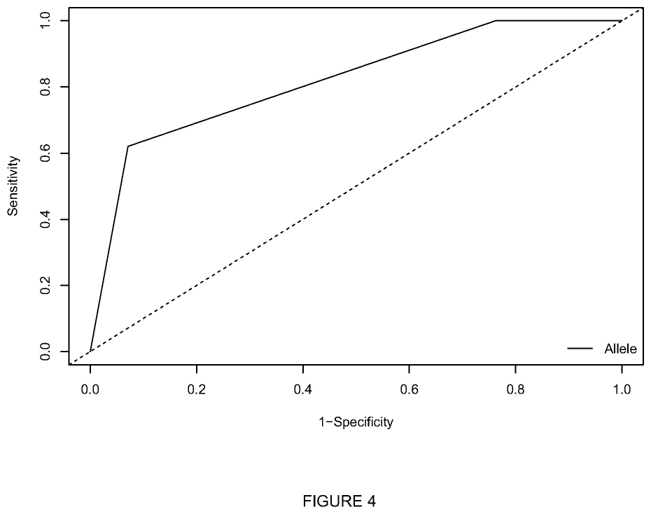

FIG. 4: Receiver operating characteristic (ROC) curve of genotypic test for rs4442490 at predicting gabapentin pain response. Area under curve 0.82 (95% CI 0.74-0.90).

### METHODOLOGY

This study is a planned secondary analysis of the GaPP2 trial, a UK-based multicentre, randomised, placebo-controlled trial of gabapentin versus placebo in women with chronic pelvic pain. Trial protocol including ethical approval and exclusion and inclusion criteria are published in full elsewhere. (17) All participants were women aged 18-50 years of age, were using or willing to use contraception to avoid pregnancy and had no obvious pelvic pathology at laparoscopy which must have taken place at least 2 weeks and less than 36 months prior to consent. Participants were randomly assigned to gabapentin (titrated to maximum dose 2700 mg daily) or placebo for 16 weeks. At inclusion to the trial, participants were asked to provide a saliva sample for inclusion in this study.

### Phenotype Definition

Cases were categorised into two phenotypes assessing the analgesic response to gabapentin and the degree of intolerance (side-effect burden). Gabapentin analgesic response was defined categorically using the coprimary outcomes of the GaPP2 trial: worst and average pain. Gabapentin ‘responders’ were defined as having reported a ≥30% reduction in worst and/or average pain scores on a numerical rating scale (1-10) after 12 weeks of gabapentin exposure. Gabapentin intolerance was also categorised categorically using a cut-off of ≥3 side-effects reported during the study period. Cases were categorised into ≥3 side-effects and <3 side-effects.

### DNA Extraction and Genotyping

Saliva samples were collected using Isohelix (UK) saliva collection kits following manufacturers instructions. Sample quality was assessed using a Quant-iT Picogreen dsDNA Assay kits and a NanoDrop Spectrophotometer (Themofisher, USA). Samples (n=93) were genotyped at Edinburgh Clinical Research Facility using an Illumina Global Screening Array on an Illumina iScan (Illumina Inc, San Diego, USA). Genotypes were called using GenomeStudio (v2.0.3) and genotypes with a GenCall (v6.3.0) cut-off of <0.15 were declared as missing.

### Quality Control and Imputation

We performed quality control using PLINK v1.07 (18), excluding samples with discordant sex information, outlying missingness (≥5%) and heterozygosity (>2 standard deviations from the mean). Relatedness was assessed by testing pairwise identity-by-decent (IBD) and one individual from each related pair (pi-hat>0.185) was excluded. Principal components analysis was conducted, and individuals of divergent ancestry were excluded. Variant quality control was applied and SNPs with genotypic call rates ≤95%, Harvey-Weinberg equilibrium p≤0.00001, and minor allele frequency ≤5% were excluded. A strict (≤5%) minor allele frequency cut-off was adopted to due to the small sample size in this study. Genotypes were pre-phased using SHAPEIT (19) and imputation of genotypes was performed using the Haplotype Reference Consortium (HRC) reference panel via the Michigan Imputation Server (20). Post-imputation quality control was performed and SNPs with poor imputation quality (information score≤0.4) and minor allele frequency ≤5% were excluded.

### Association Analyses and Post-GWAS

Genome-wide association was performed using 2 phenotypes of interest: gabapentin analgesic response and gabapentin intolerance. Frequentist association analysis was conducted in SNPTEST v2.5.4-beta1 (21) including autosomes and chromosome X. No co-variates were used. A standard genome-wide significant threshold of p<5×10-8 was adopted to identify significant genome-wide associations. Candidate gene analysis was performed for CACNA1C, KCNQ2, KCNQ3, KCNQ5, OCT2, OCTN1, COMT, ADRB2, NRG1, NRG1-IT1, NRG2, NRG4, ERBB3, ERBB4, KRAS, HSP90AA1, EGFR, MAPK3, HRAS. These genes were selected due to either their relevance to gabapentin pharmacology or though network analysis with NRG3. Statistical testing was performed for SNPs within each gene plus 500 kb upstream and downstream with a statistical significance identified using a p with a Bonferroni multiple testing correction. Regional association plots for loci were produced using LocusZoom (22). Loci were assigned to mapped genes using FUMA v1.3.8 (24) and the GENE2FUNC tool was used to create bulk gene expression heatmaps using GTEx v8.

Analysis of Lead SNPs Association with Brain Imaging Phenotypes in UK Biobank

To provide more insight into potential central neurological pathways associated with the genetic signatures of gabapentin response, we looked up genome-wide significant loci lead SNPs in phenome-wide association study brain imaging-derived phenotypes within the UK Biobank (29). Brain imaging phenotypes were provided by the Oxford Brain Imaging Genetic (BIG40) web server (30) and examined using the PheWeb interface (31). BIG40 is derived from the 40 k release of UK Biobank, using data from 33,224 people, of whom 17,411 are genetic females, with mean age 64.4 years (7.5 SD). Imaging pre-processing pipelines have previously been described in detail (32, 33). Derived phenotypes include regional measures of structural volume, cortical thickness, cortical grey-white contrast; white matter tract mean orientation and orientation dispersion; and resting-state fMRI node amplitude and inter-node correlation. Phenotypes achieving Bonferroni-corrected significance of p<0.05 across the 3935 imaging phenotypes were reported.

Comparing Gabapentin Responders and Non-Responders According to Lead SNP Association Using fMRI

Within the sample taking part in the gabapentin RCT, a small number also took part in a mechanistic fMRI study, undergoing MRI brain scans before and after 12 weeks of treatment. Each scan comprised structural imaging, and a 10 m50 s punctate task, during which they received 39 applications of a 300 g Touch Test von Frey filament (6.65 mm) to their lower abdomen, 10 cm above the superior edge of the pubic bone. Punctate task fMRI pre-processing is described in the supplementary materials. For those for whom both genetic and imaging data was available, the gabapentin pre- and post-treatment scans were compared. The first-level contrast of post-treatment>pre-treatment punctate vs rest was taken into a random effects analysis comparing SNP variants, with clusters achieving a whole-brain, FWE-corrected significance of p<0.05 being reported.

### EXAMPLES

93 participants provided samples for genotyping. A study flowchart is of participants is presented in FIG. 3. Following standard quality control and imputation, 82 samples remained, and 5522729 SNPs were tested for association. Baseline characteristics of participants including baseline average and worst pain were similar among analgesic response and side-effect phenotypes (Table 2).

TABLE 2
Characteristics of participants per phenotype.

| | Pain responder | | Pain non-responder | | |
|---|---|---|---|---|---|
| | (n = 31) | | (n = 46) | | p |
| Age (years) | 32.4 | (8.2) | 29.7 | (7.5) | 0.15 |

| | | | |
|---|---|---|---|
| BMI (kg/m²) | 28.4 (7.6), 30 | 26.3 (4.8), 45 | 0.20 |

Average pain (NRS)

| | | | | | |
|---|---|---|---|---|---|
| Baseline | 5.2 | (1.7) | 5.6 | (1.6) | 0.41 |
| End of study | 2.5 | (1.4) | 5.8 | (1.5) | <0.001 |

Baseline worst pain (NRS)

| Baseline | 9 | [1.5] | 9 | [1] | 0.60 |
|---|---|---|---|---|---|
| End of study | 6 | [3.5] | 9 | [1] | <0.001 |
| | ≥3 side-effects (n = 53) | | <3 side-effects (n = 23) | | |
| Age (years) | 31.3 | (7.3) | 29.2 | (8.3) | 0.30 |
| BMI (kg/m²) | 27.3 (6.4), 52 | | 27.8 | (5.6) | 0.71 |
| Baseline average pain (NRS) | 5.7 | (1.6) | 5.4 | (1.6) | 0.40 |
| Baseline worst pain (NRS) | 9 | [1] | 9 | [2] | 0.36 |

Mean (SD); median [IQR], n where n is different to the total number of participants.

Minimal evidence of population stratification was observed for gabapentin pain response (λ=1.046) or gabapentin side-effect burden (λ=1.033). For gabapentin pain response, 6 SNPs in one locus reached genome-wide significance (FIG. 1). The lead SNP for gabapentin pain response was chr10:84685978, an intron variant located in Neuregulin 3 (NRG3) at 10q23.1 (p=2.11×10-8; OR=18.82 (95% CI 4.86-72.83). Five neighbouring SNPs in linkage disequilibrium (LD) with chr10:84685978 (r2>0.6) also reached genome-wide significance (Table 1). No SNPs reached genome-wide significance for gabapentin side-effect burden. No SNPs significant associations were identified in candidate gene analysis for gabapentin analgesic response or side-effect burden.

All 6 SNPs comprising the genome-wide significant locus in NRG3 were imputed, however with high imputation quality scores (>0.97). Three of these SNPs (chr10:84685978, chr10:84690540 and chr10:84688752) were multiallelic allelic SNPs and the remaining three were biallelic. 28 genotyped SNPs were located within 100 kb upstream and downstream of the lead SNP (chr10:84685978), with three of these genotyped SNPs in high LD (r2>0.6) with chr10:84685978 for which intensity plots were generated which showed accurate genotype calling in discrete groups.

Functional mapping of candidate loci was performed using FUMA which mapped to 31 genes using one genomic risk locus (chr10:84685978). A heatmap of bulk gene expression in GTEx v8 for mapped genes was produces using the GENE2FUNC tool, showing NRG3 is expressed predominantly across brain tissues (FIG. 2).

No eQTL data was available in GTEx v8 for the 6 SNPs that reached genome-wide significance.

Analysis of Lead SNP Association with Brain Imaging Phenotypes in UK Biobank

A phenome-wide association study of the lead SNP rs4442490 within UK Biobank's 40 k dataset reveals Bonferroni-corrected significant differences across a range of metrics (see Table 3). Structural measures comprised volumetric and grey-white contrast variation in orbitofrontal regions. DTI-tractography implicated the superior longitudinal fasciculus. Resting state networks comprised several regions, dominated by connections between orbitofrontal/insula areas and posterior midline/parietal regions.

TABLE 3
Brain imaging-derived phenome-wide association study for rs4442490.

| Modality | Phenotype | β | p |
|---|---|---|---|
| Diffusion- | Weighted-mean orientation dispersion | 0.031 | 1.1 × 10⁻⁴ |
| weighted | index in tract left superior longitudinal | | |
| tractography | fasciculus | | |
| Resting-state | ICA100 edge 309 | −0.029 | 2.6 × 10⁻⁴ |
| fMRI | Node 9 | STG, PHG, OFC, mediodorsal thalamus | |
| | Node 26 | R posterior ITG, pre/postcentral gyri, insula, | |
| | | OFC | |
| | ICA100 edge 549 | 0.029 | 3.5 × 10⁻⁴ |
| | Node 21 | IFG, medial MFG/SFG, subgenual ACC, | |
| | | ventral striatum, hippocampus | |
| | Node 34 | SPL, precuneus, pre/postcentral gyri | |
| | ICA100 edge 713 | −0.028 | 5.6 × 10⁻⁴ |
| | Node 10 | MTG, fusiform gyrus, PHG, PCC | |
| | Node 39 | Precuneus, SOG | |
| T1-weighted | Volume R amygdala - cortical nucleus | −0.027 | 8.5 × 10⁻⁴ |
| structural | Grey-white contrast L medial OFC | 0.026 | 9.4 × 10⁻⁴ |
| | Volume R medial orbital sulcus | 0.025 | 1.4 × 10⁻³ |
| | (olfactory) | | |

Resting-state fMRI ICA 100 refers to a 100-dimension parcellation by independent components analysis.
The partial correlation between each parcellation or “node” defines each “edge”.
The dominant anatomical regions within each node are listed.
ACC: Anterior cingulate cortex; IFG: Inferior frontal cortex; ITG: Inferior temporal gyrus; MFG: Middle frontal cortex; MTG: middle temporal gyrus; OFC: Orbitofrontal cortex; PCC: Posterior cingulate cortex; PHG: Parahippocampal gyrus; SFG: superior frontal cortex; SOG: Superior occipital cortex; SPL: Superior parietal lobule; STG: Superior temporal gyrus.

Comparing Gabapentin Responders and Non-Responders According Lead SNP Association Using fMRI Brain

Identifying GaPP2 participants with both genomic and neuroimaging data revealed two people who were classified as gabapentin responders—both of whom were rs4442490 TT homozygotes—and five gabapentin non-responders, who were all either TG or GG. Given the small amount of available data, an exploratory analysis was performed by which fMRI responses to punctate stimuli were compared between the TT responder and TG/GG non-responder groups. A full factorial model with pre- and post-treatment as a within-subjects effect, and responder status as a between-subjects effects, revealed significant activation within several brain regions, corrected for multiple comparisons across the whole brain volume (see Table 4). In all cases, the responders showed a decrease in punctate activation after treatment with gabapentin.

TABLE 4
Comparing rs4442490 TT responders and TG/GG non-responders: Brain regions showing
significant fMRI activation changes in response to punctate stimuli following treatment
with gabapentin. In all cases, there was a significant decrease in activation post-
gabapentin in the responder group, but not the non-responder group.

| | MNI | | | Cluster P | UK Biobank |
|---|---|---|---|---|---|
| Region | coordinates | Z | kE | (FWE-corrected) | node (edge) |
| R Calcarine cortex | 20 −86 10 | 4.56 | 34 | 0.007 | — |
| L lateral OFC/IFG | −42 38 −14 | 4.54 | 126 | <0.001 | 39 (713) |
| Lingual/calcarine cortex | 0 −84 0 | 4.45 | 50 | <0.001 | — |
| R supramarginal cortex | 54 −24 36 | 4.39 | 33 | 0.009 | 26 (309) |
| R middle/superior occipital | 32 −80 26 | 4.25 | 39 | 0.003 | — |
| cortex | | | | | |
| R superior parietal lobule | 22 −50 52 | 4.19 | 47 | 0.001 | 34 (549) |
| L anterior/medial OFC | −22 48 −16 | 4.11 | 26 | 0.037 | 21 (549) |
| R anterior/lateral OFG | 38 56 −8 | 4.08 | 25 | 0.045 | — |
| R anterior MFG/frontal pole | 32 52 8 | 3.83 | 63 | <0.001 | — |

### REFERENCES

1. Latthe P, Latthe M, Say L, Gülmezoglu M, Khan K S. WHO systematic review of prevalence of chronic pelvic pain: a neglected reproductive health morbidity. BMC public health. 2006 December; 6 (1): 1-7.
2. Daniels J P, Khan K S. Chronic pelvic pain in women. Bmj. 2010 Oct. 5; 341.
3. Daniels J, Gray R, Hills R K, Latthe P, Buckley L, Gupta J, Selman T, Adey E, Xiong T, Champaneria R, Lilford R. Laparoscopic uterosacral nerve ablation for alleviating chronic pelvic pain: a randomized controlled trial. Jama. 2009 Sep. 2; 302 (9): 955-61.
4. Iannetti G D, Zambreanu L, Wise R G, Buchanan T J, Huggins J P, Smart T S, Vennart W, Tracey I. Pharmacological modulation of pain-related brain activity during normal and central sensitization states in humans. Proceedings of the National Academy of Sciences. 2005 Dec. 13; 102 (50): 18195-200.
5. Whitaker L H, Reid J, Choa A, McFee S, Seretny M, Wilson J, Elton R A, Vincent K, Horne A W. An exploratory study into objective and reported characteristics of neuropathic pain in women with chronic pelvic pain. PLOS One. 2016 Apr. 5; 11 (4): e0151950.
6. Montastruc F, Loo S Y, Renoux C. Trends in first gabapentin and pregabalin prescriptions in primary care in the United Kingdom, 1993-2017. Jama. 2018 Nov. 27; 320 (20): 2149-51.
7. Horne A W, Vincent K, Hewitt C A, Middleton L J, Koscielniak M, Szubert W, Doust A M, Daniels J P, Abdi S, Acharya S, Al-Inizi S. Gabapentin for chronic pelvic pain in women (GaPP2): a multicentre, randomised, double-blind, placebo-controlled trial. The Lancet. 2020 Sep. 26; 396 (10255): 909-17.
8. Hewitt C A, Vincent K, Middleton L J, Romaniuk L, Koscielniak M, Doust A M, Birch J, Whalley H, Daniels J P, Horne A W. Results of the mechanistic substudy. In Gabapentin to reduce pain in women aged between 18 and 50 years with chronic pelvic pain: the GaPP2 RTC. Efficacy and Mechanism Evaluation. 2020 November, No. 7.7.
9. Mogil J S. Pain genetics: past, present and future. Trends in genetics. 2012 Jun. 1; 28 (6): 258-66.
10. Till S R, As-Sanie S, Schrepf A. Psychology of chronic pelvic pain: prevalence, neurobiological vulnerabilities, and treatment. Clinical obstetrics and gynecology. 2019 March; 62 (1): 22.
11. Veluchamy A, Hebert H L, Meng W, Palmer C N, Smith B H. Systematic review and meta-analysis of genetic risk factors for neuropathic pain. Pain. 2018 May 1; 159 (5): 825-48.
12. Farrell S F, Campos Al, Kho P F, de Zoete R M, Sterling M, Rentería M E, Ngo T T, Cuéllar-Partida G. Genetic basis to structural grey matter associations with chronic pain. Brain. 2021 December; 144 (12): 3611-22.
13. Yamamoto, P. A., Benzi, J. R., Azeredo, F. J., Dach, F., lanhez Junior, E., Zanelli, C. F. and de Moraes, N. V., 2019. Pharmacogenetics-based population pharmacokinetic analysis of gabapentin in patients with chronic pain: Effect of October 2 and OCTN 1 gene polymorphisms. Basic & Clinical Pharmacology & Toxicology, 124 (3), pp. 266-272.
14. Lloyd R A, Hotham E, Hall C, Williams M, Suppiah V. Pharmacogenomics and patient treatment parameters to opioid treatment in chronic pain: a focus on morphine, oxycodone, tramadol, and fentanyl. Pain Medicine. 2017 Dec. 1; 18 (12): 2369-87.
15. Haerian B S, Haerian M S. OPRM1 rs1799971 polymorphism and opioid dependence: evidence from a meta-analysis. Pharmacogenomics. 2013 May; 14 (7): 813-24.
16. Hung C C, Chen P L, Huang W M, Tai J J, Hsieh T J, Ding S T, Hsieh Y W, Liou H H. Gene-wide tagging study of the effects of common genetic polymorphisms in the a subunits of the GABAA receptor on epilepsy treatment response. Pharmacogenomics. 2013 November; 14 (15): 1849-56.
17. Vincent K, Baranowski A, Bhattacharya S, Birch J, Cheong Y, Cregg R, Daniels J, Hewitt C A, Macfarlane G J, Middleton L, Szubert W. GaPP2, a multicentre randomised controlled trial of the efficacy of gabapentin for the management of chronic pelvic pain in women: study protocol. BMJ open. 2018 Jan. 1; 8 (1): e014924.
18. Purcell S, Neale B, Todd-Brown K, Thomas L, Ferreira M A, Bender D, Maller J, Sklar P, De Bakker P I, Daly M J, Sham P C. PLINK: a tool set for whole-genome association and population-based linkage analyses. The American journal of human genetics. 2007 Sep. 1; 81 (3): 559-75.
19. Delaneau O, Marchini J, Zagury J F. A linear complexity phasing method for thousands of genomes. Nature methods. 2012 February; 9 (2): 179-81.
20. Das S, Forer L, Schönherr S, Sidore C, Locke A E, Kwong A, Vrieze S I, Chew E Y, Levy S, McGue M, Schlessinger D. Next-generation genotype imputation service and methods. Nature genetics. 2016 October; 48 (10): 1284-7.
21. Marchini J, Howie B, Myers S, McVean G, Donnelly P. A new multipoint method for genome-wide association studies by imputation of genotypes. Nature genetics. 2007 July; 39 (7): 906-13.
22. Watanabe K, Taskesen E, Van Bochoven A, Posthuma D. Functional mapping and annotation of genetic associations with FUMA. Nature communications. 2017 Nov. 28; 8 (1): 1-1.

1. A method of identifying subjects likely to respond to treatment with gabapentin, said method comprising assessing the genetic profile of a subject considered for a gabapentin-based therapy and determining, on the basis of the genetic profile, whether or not to administer the subject gabapentin, wherein assessing the genetic profile of a subject comprises the identification of one or more genetic variation(s).

2. The method according to claim 1, wherein assessing the genetic profile comprises identification of one or more genetic variation(s) on chromosome 10q23.

3. The method according to claim 1, wherein said one or more genetic variation(s) comprises:
(i) one or more amino acid substitution(s);
(ii) one or more amino acid deletion(s);
(iii) one or more amino acid addition(s)/insertion(s);
(iv) one or more amino acid/sequence inversions; and/or
(v) one or more amino acid/sequence duplications.

4. The method according to claim 1, wherein said one or more genetic variations(s) comprises one or more single nucleotide polymorphism(s).

5. The method according to claim 4, wherein said one or more single nucleotide polymorphism(s) is located on chromosome 10q23.

6. The method according to claim 4, wherein said one or more single nucleotide polymorphism(s) is located on chromosome 10q23.1.

7. The method according to claim 4, wherein said one or more single nucleotide polymorphism(s) is located in the Neuregulin 3 (NRG3) gene.

8. The method according to claim 4, wherein said one or more single nucleotide polymorphism(s) is in linkage disequilibrium with the Neuregulin 3 (NRG3) gene.

9. The method according to claim 4, wherein said one or more single nucleotide polymorphism(s) is located at position(s) selected from the group consisting of:
(i) chr10:84685978;
(ii) chr10:84690540;
(iii) chr10:84681635;
(iv) chr10:84685529;
(v) chr10:84688752;
(vi) chr10:84682591; and/or
(vii) single nucleotide polymorphism(s) or genetic variations in linkage disequilibrium with any of (i)-(vi).

10. The method according to claim 4, wherein said one or more single nucleotide polymorphism(s) is selected from:
(i) T at chr10:84685978;
(ii) T at chr10:84690540;
(iii) T at chr10:84681635;
(iv) A at chr10:84685529;
(v) A at chr10:84688752;
(vi) C at chr10:84682591 and/or
(vii) single nucleotide polymorphism(s) or genetic variations in linkage disequilibrium with any of (i)-(vi).

11. The method according to claim 8, wherein the measure of linkage disequilibrium of the one or more single nucleotide polymorphism(s) is r²>0.6 and/or located within 100 kb of one or more single nucleotide polymorphism(s) located on chromosome 10q23, located on chromosome 10q23.1, located in the Neuregulin 3 (NRG3) gene, in linkage disequilibrium with the Neuregulin 3 (NRG3) gene,
located at position(s) selected from the group consisting of:
(i) chr10:84685978;
(ii) chr10:84690540;
(iii) chr10:84681635;
(iv) chr10:84685529;
(v) chr10:84688752;
(vi) chr10:84682591; and/or
(vii) single nucleotide polymorphism(s) or genetic variations in linkage disequilibrium with any of (i)-(vi), and/or
selected from:
(i) T at chr10:84685978;
(ii) T at chr10:84690540;
(iii) T at chr10:84681635;
(iv) A at chr10:84685529;
(v) A at chr10:84688752;
(vi) C at chr10:84682591 and/or
(vii) single nucleotide polymorphism(s) or genetic variations in linkage disequilibrium with any of (i)-(vi).

12. The method according to claim 1, wherein the subject suffers from chronic pain.

13. The method according to claim 1, wherein the subject suffers from chronic pelvic pain.

14. The method according to claim 1, wherein the subject is female.

15. A kit for the detection of the presence of one or more genetic variation(s) of claim 9, wherein the kit comprises one or more pairs of oligonucleotide primers for amplifying said one or more genetic variations and one or more probes for detecting the resulting amplicon.

16. A method of identifying subjects likely to respond to treatment with gabapentin, said method comprising assessing the genetic profile of a subject considered for a gabapentin-based therapy to determine whether or not they harbour one or more of the following SNP alleles:
(i) T at chr10:84685978;
(ii) T at chr10:84690540;
(iii) T at chr10:84681635;
(iv) A at chr10:84685529;
(v) A at chr10:84688752; and/or
(vi) C at chr10:84682591;
wherein, if the subject harbours one or more of these SNP alleles, they may respond to gabapentin and should be recommended for gabapentin therapy.

17. The method according to claim 9, wherein the measure of linkage disequilibrium of the one or more single nucleotide polymorphism(s) is r2>0.6 and/or located within 100 kb of one or more single nucleotide polymorphism(s) located on chromosome 10q23, located on chromosome 10q23.1, located in the Neuregulin 3 (NRG3) gene, in linkage disequilibrium with the Neuregulin 3 (NRG3) gene,
located at position(s) selected from the group consisting of:
(i) chr10:84685978;
(ii) chr10:84690540;
(iii) chr10:84681635;
(iv) chr10:84685529;
(v) chr10:84688752;
(vi) chr10:84682591; and/or
(vii) single nucleotide polymorphism(s) or genetic variations in linkage disequilibrium with any of (i)-(vi), and/or
selected from:
(i) T at chr10:84685978;
(ii) T at chr10:84690540;
(iii) T at chr10:84681635;
(iv) A at chr10:84685529;
(v) A at chr10:84688752;
(vi) C at chr10:84682591 and/or
(vii) single nucleotide polymorphism(s) or genetic variations in linkage disequilibrium with any of (i)-(vi).

18. The method according to claim 10, wherein the measure of linkage disequilibrium of the one or more single nucleotide polymorphism(s) is r2>0.6 and/or located within 100 kb of one or more single nucleotide polymorphism(s) located on chromosome 10q23, located on chromosome 10q23.1, located in the Neuregulin 3 (NRG3) gene, in linkage disequilibrium with the Neuregulin 3 (NRG3) gene,
located at position(s) selected from the group consisting of:
(i) chr10:84685978;
(ii) chr10:84690540;
(iii) chr10:84681635;
(iv) chr10:84685529;
(v) chr10:84688752;
(vi) chr10:84682591; and/or
(vii) single nucleotide polymorphism(s) or genetic variations in linkage disequilibrium with any of (i)-(vi), and/or
selected from:
(i) T at chr10:84685978;
(ii) T at chr10:84690540;
(iii) T at chr10:84681635;
(iv) A at chr10:84685529;
(v) A at chr10:84688752;
(vi) C at chr10:84682591 and/or
(vii) single nucleotide polymorphism(s) or genetic variations in linkage disequilibrium with any of (i)-(vi).
